(19) United States
(12) Patent Application Publication
(10) Pub. No.: US 2010/0018214 A1
(43) Pub. Date: Jan. 28, 2010

(54) Energy Production from Algae in Photo Bioreactors Enriched with Carbon Dioxide

(76) Inventor: Eliezer Halachmi Katchanov, Oranit (IL)

Correspondence Address: The Law Office of Michael E. Kondoudis, 888 16th Street, N.W., Suite 800, Washington, DC, 20006, US

(21) Appl. No.: 12/177,298

(22) Filed: Jul. 22, 2008

(51) Int. Cl. F02C 3/00; C12M 1/42

(52) U.S. Cl. 60/772; 435/292.1

(57) ABSTRACT

A system and method for energy production. The system includes a kiln for burning limestone and dolomite into clinker, two photo bioreactors for growing algae and pipeworks for feeding co₂ from the kiln and from limestone and dolomite to the photo bioreactors. The CO₂ intensifies the growth of algae. The system further includes a harvester for harvesting the algae and an energy producing module for producing energy from the algae biomass.

### FIELD OF THE INVENTION

The present invention generally relates to the field of energy production. More particularly, the present invention relates to energy production from algae biomass.

### BACKGROUND OF THE INVENTION

A presentation titled “Experimental study on sequestrating of CO₂ in the true flue gas by ammonia spray and producing NH₄HCO₃ by Zhang, Y., Li, Z.-Z., Li, C.-Z., Dong, J.-X. and Wang, Y. from the National Power Plant Combustion Engineering Research Center, Shenyang, China, is incorporated herein by reference in its entirety.

U.S. Pat. No. 6,477,841, which is incorporated herein by reference in its entirety, discloses a closed cycle power plant for conversion of solar to electrical energy has water tank for growing macroalgae and fluidized bed combustion chamber for combusting macroalgae in presence of oxygen and carbon dioxide.

EP0561436B1, which is incorporated herein by reference in its entirety, discloses a process for making cement by preheating the raw meal in a cyclone heater, calcining the preheated raw meal, burning the calcined raw meal in a rotary kiln, cooling the cement clinker formed in the rotary kiln and grinding the cement klinker.

US20020194782, which is incorporated herein by reference in its entirety, discloses an integrated biomass gasification and fuel cell system wherein the electrochemical reaction in the fuel cell is effected by providing the reactant gases from a gasifier.

WO2000057105, which is incorporated herein by reference in its entirety, discloses a closed cycle power plant for the conversion of solar energy stored by photosynthesis to electrical energy, comprising a body of water for growing macroalgae therein, and a fluidized bed combustion chamber for at least partial combustion of partially dried macroalgae having a water content of up to 60% wt/wt, the combustion being carried out in an artificial atmosphere of oxygen and carbon dioxide.

WO1995024548, which is incorporated herein by reference in its entirety, discloses an internal combustion engine comprising combustion chamber means, inlet track means, for directing air into the combustion chamber means, and fuel induction means, for supplying powdered fuel to be burned in the combustion chamber means, in which the fuel induction means are arranged for supplying the powdered fuel into the inlet track means, so that the so supplied powdered fuel forms a substantially homogenous fuel/air mixture for ignition in the combustion chamber means, during the engine's operation.

U.S. Pat. No. 5,659,977, which is incorporated herein by reference in its entirety, discloses an integrated plant including a microalgae production plant for growing, harvesting and drying algae and a fossil fuel-motor-generator plant producing electrical energy. A fossil fuel engine produces hot exhaust gas from which sensible heat dries the algae. The drying may be direct from the exhaust gas or may be indirect with the hot exhaust gas exchanging sensible heat with a recirculating stream of inert gas. Carbon dioxide from the exhaust gas is recovered for use as a nutrient in the microalgae production plant. Electrical energy from the generator is used to drive motors and/or produce artificial illumination and/or drive pumps, motors and controls in the microalgae production plant.

U.S. Pat. No. 6,477,841, which is incorporated herein by reference in its entirety, discloses a method for the conversion of solar energy stored by photosynthesis to electrical energy, utilizing a closed cycle power plant comprising providing a body of water for growing macroalgae therein, and providing a fluidized bed combustion chamber for at least partial combustion of partially dried macroalgae having a water content of up to 60% wt/wt, the combustion being carried out in an artificial atmosphere of oxygen and carbon dioxide.

WO2007047805, which is incorporated herein by reference in its entirety, discloses a device and method for carbon dioxide sequestering involving the use of a photo-bioreactor with Light Emitting Diodes (LED's) for the cost-effective photo-fixation of carbon dioxide (CO₂). This device and method is useful for removing undesirable carbon dioxide from waste streams.

### BRIEF SUMMARY

The present invention provides systems and methods for energy production. One method comprises the stages: (i) sustaining and regulating an algae culture in at least two photo bioreactors, (ii) introducing CO₂ from at least one kiln which is arranged for burning limestone and dolomite into clinker, (iii) harvesting algae from the bioreactors, (iv) producing energy from the biomass of harvested algae. One system comprises at least one kiln for burning limestone and dolomite into clinker, at least two photo bioreactors for growing algae; a first pipework for feeding CO₂ from the kiln to a first photo bioreactor; a second pipework for feeding CO₂ released from burning limestone and dolomite to a second photo bioreactor, a harvester for taking algae out of the photo bioreactors, and means for producing energy from the biomass of the algae taken out of the photo bioreactors. In embodiments, CO₂ from the kiln is used to intensify the growth of the algae in the photo bioreactors. A large excess of algae is produced by this synergetic arrangement, and this access can be used for electric energy production, water desalination and additional energy consuming processes.

### BRIEF DESCRIPTION OF THE DRAWINGS

The subject matter regarded as the invention will become more clearly understood in light of the ensuing description of embodiments herein, given by way of example and for purposes of illustrative discussion of the present invention only, with reference to the accompanying drawings (Figures, or simply “FIGS.”), wherein:

FIG. 1 is a block diagram illustrating a system for energy production, according to some embodiments of the invention;

FIG. 2 is a block diagram illustrating influxes to and effluxes from container with water and algae, according to some embodiments of the invention;

FIG. 3 is a flowchart illustrating a method of producing energy, according to some embodiments of the invention;

FIG. 4 is a flowchart illustrating a method of harvesting algae from the bioreactors, according to some embodiments of the invention;

FIG. 5 is a flowchart illustrating optional stages in a method of sustaining and regulating an algae culture in photo bioreactors, according to some embodiments of the invention;

FIG. 6 is a block diagram illustrating a system for producing energy by growing algae in a large excess utilizing CO₂ emitted during the production of clinker and quicklime, according to some embodiments of the invention.

### DETAILED DESCRIPTIONS OF SOME EMBODIMENTS OF THE INVENTION

The present invention includes a system and method for energy production utilizing CO₂ emitted from burning limestone and dolomite to intensify the growth of algae in photo bioreactors. The algae biomass is then utilized to generate energy.

The term “Limestone” as used herein in this application comprises calcite, dolomite and a combination thereof.

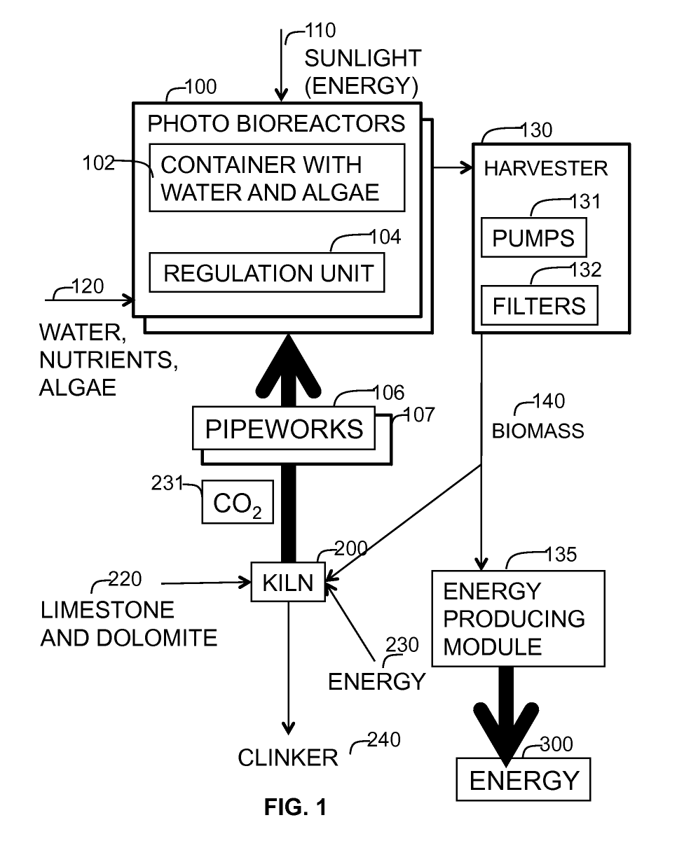

FIG. 1 is a block diagram illustrating a system for energy production, according to some embodiments of the invention. The system comprises at least one kiln 200 for burning limestone and dolomite 220 into clinker 240. Kiln 200 is heated with energy 230 and burning limestone and dolomite 220 produces large amounts of CO₂ 231. The system further comprises at least two one photo bioreactors 100 for growing algae, comprising a container 102 with water and algae and a regulation unit 104. Photo bioreactors 100 receives water, nutrients and algae 120, CO₂ 231 as well as sunlight 110 and these are regulated by regulation unit 104.

The system further comprises a first pipework 106 for feeding CO₂ from kiln 200 to one of photo bioreactors 100 and a second pipework 107 for feeding CO₂ released from burning limestone and dolomite 220 to another of photo bioreactors 100. CO₂ 231 comprises both CO₂ from kiln 200 and CO₂ released from burning limestone and dolomite 220 as well as further possible. CO₂ 231 intensifies the growth of the algae in photo bioreactors 100. The system further comprises a harvester 130 for taking algae out of photo bioreactors 100 and an energy producing module 135 for producing energy 300 from the biomass 140 of the algae taken out of the photo bioreactors. Some of biomass 140 may be used to heating kiln 200.

According to some embodiments of the invention, one photo bioreactor 100 is fed by CO₂ 231 released from burnt algae cells in the flue gas emitted from the kiln 200, and the second of photo bioreactors 100 is fed with CO₂ 231 emitted from the limestone and dolomite 220. The two pipe works are for feeding CO₂ 231 from kiln 200 to the photo bioreactors 100. The two pipe works comprise one for the flue gas, and one for the CO₂ 231 released from the limestone and the dolomite 220. CO₂ 231 from kiln 200 is used to intensify the growth of the algae in the photo bioreactors 100. A large excess of algae is produced by this synergetic arrangement, and this excess can be used for electric energy production, water desalination and additional energy consuming processes.

According to some embodiments of the invention, substantially all CO₂ released by burning limestone and dolomite into clinker in kiln 200 is introduced into photo bioreactors 100. According to some embodiments of the invention, at least one analyzer may be connected to a control system, and arranged to keep the CO₂ concentration in photo bioreactors 100 at an optimal level (e.g. 3% concentration of CO₂ in the inflowing air). According to some embodiments of the invention, the system may further comprise a bubbler for introducing CO₂ into photo bioreactors 100.

According to some embodiments of the invention, harvester 130 may comprise pumps 131 for pumping water out of photo bioreactors 100, filters 132 for extracting algae out of the pumped water and a drier for drying the extracted algae. The a drier may comprise kiln 200 and water may be fed back into photo bioreactors 100. Energy producing module 135 may comprise kiln 200.

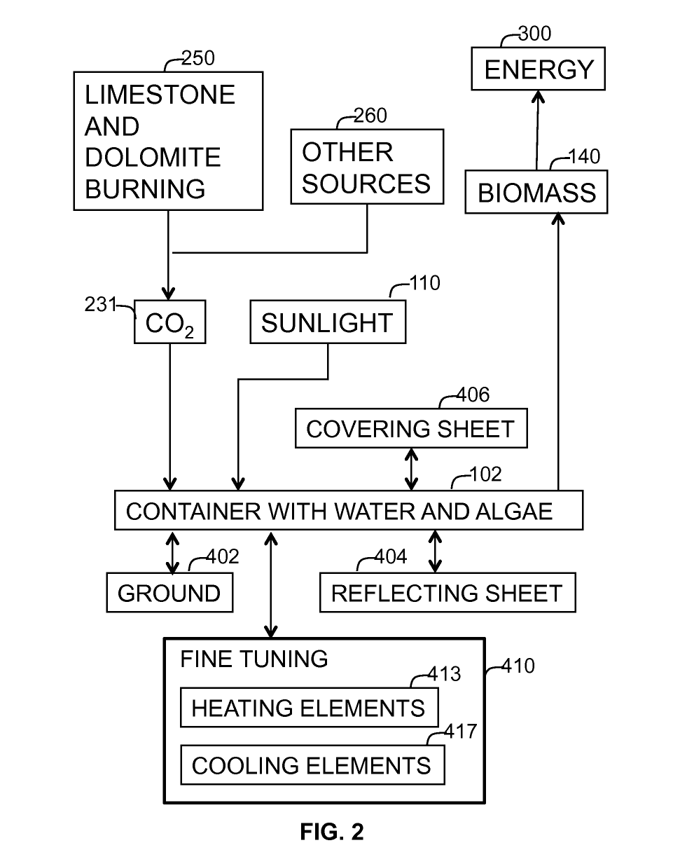

FIG. 2 is a block diagram illustrating influxes to and effluxes from container with water and algae 102, according to some embodiments of the invention. Energy from sunlight 110 falling upon container 102 may be attenuated or intensified by placing reflecting sheets 404 below container 102 and covering sheets 406 above container 102. Water temperature may be further controlled by warm and cold water coming from and flowing to external sources, as well as by controlled heating elements 413 and cooling elements 417 in a fine tuning unit 410 inside or near container 102. CO₂ 231 may be supplied to container 102 from limestone and dolomite burning 250 (e.g. a kiln), from other sources 260 (e.g. factory flue gas, burning of biomass 140) or their combination. biomass 140 harvested from container 102 may be utilized to produce energy 300, e.g. by burning it.

According to some embodiments of the invention, algae growth rate may be increased by utilizing a CO₂ concentration of around 3% in the air supplied to bioreactors 100. CO₂ concentration may be kept by gas analyzer connected to the control system. CO₂ concentration may be kept at an optimal level in respect of different parameters of photo bioreactor 100 such as concentration of algae, water temperature, influxes and effluxes and incoming concentration of CO₂. CO₂ rich air may be bubbled into container 102.

According to some embodiments of the invention, algae may be harvested from container 102 by pumping water out of container 102 and filtering the water to extract algae from it. Harvesting rate is related to growth rate and the flow rates of water, nutrients and algae 120 into container 102. Harvested algae may be dried utilizing hot air from external process and the air may be recycled in the system. Harvested algae may be burnt in kiln 200 and the heat may be used as energy source (e.g. for producing steam). Heat from burnt algae may be utilized to produce electricity (e.g. via steam) for processes such as water desalination or other industrial processes, or for satisfying peaks in electricity consumption.

According to some embodiments of the invention, heating elements 413 may comprise heating coils in container 102, cooling elements 417 may comprise water flanges. The bottom of water container 102 may be lined with reflective material (e.g. metallic sheets, coated plastic sheets) for changing the amount of radiation absorbed by container 102. Partly reflective material may be placed on top of container 102. Container 102 may be covered to change the extent of radiation absorption and heat convection to and from container 102. The ground may be utilized to exchange heat with container 102 or with pipe work 106 in order to regulate the temperature of water moving into and from container 102. According to some embodiments of the invention, the water temperature in container 102 may be fine tuned utilizing heating elements and flanges (e.g. on container 102 or pipe work 106).

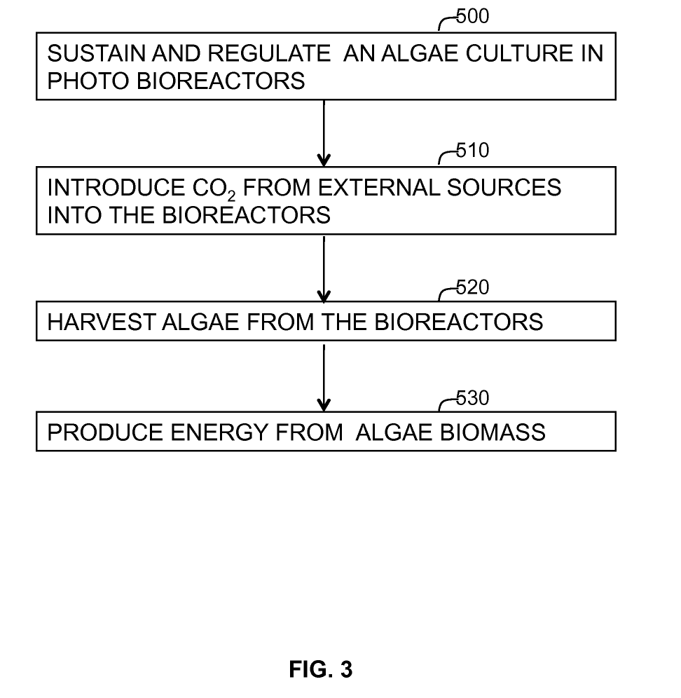

FIG. 3 is a flowchart illustrating a method of producing energy, according to some embodiments of the invention. The method comprises the stages:
Sustaining and regulating an algae culture in at least two one photo bioreactors (stage 500);
Introducing CO₂ from at least one kiln (stage 510), wherein the kiln is arranged for burning limestone and dolomite into clinker;
Harvesting algae from the bioreactors (stage 520); and
Producing energy from the biomass of harvested algae (stage 530).

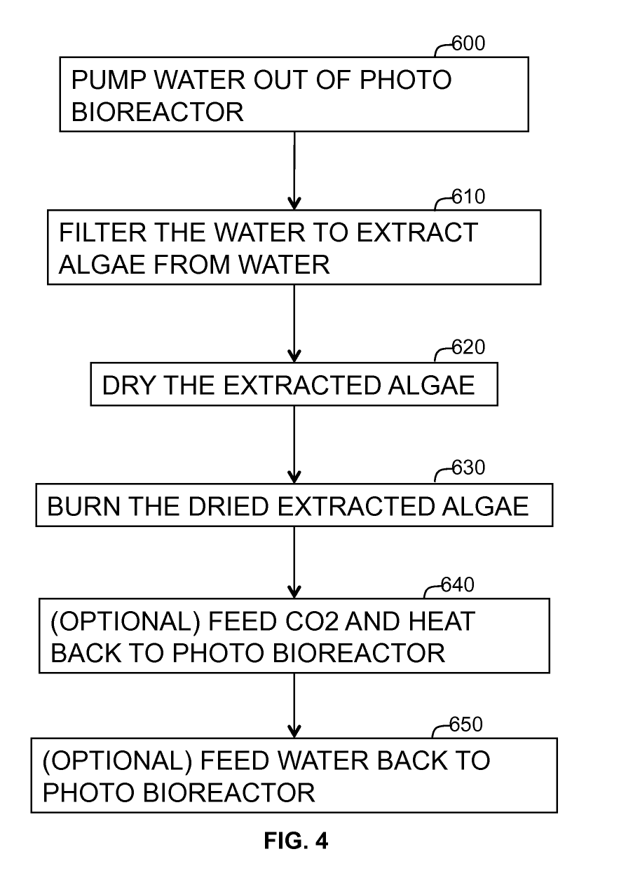

FIG. 4 is a flowchart illustrating a method of harvesting algae from the bioreactors (stage 520), according to some embodiments of the invention. The method comprises the stages:
Pumping water out of photo bioreactor (stage 600);
Filtering the water to extract algae from it (stage 610);
Drying extracted algae (stage 620); and
Burning said dried extracted algae (stage 630).

According to some embodiments of the invention, the method further comprises feeding at least part of CO₂ and heat resulting from burning dried extracted algae (stage 630) back into at least one of the photo bioreactors (stage 640).

According to some embodiments of the invention, the method further comprises feeding at least part of the water extracted during drying the extracted algae (stage 620) back into at least one of said photo bioreactors (stage 650).

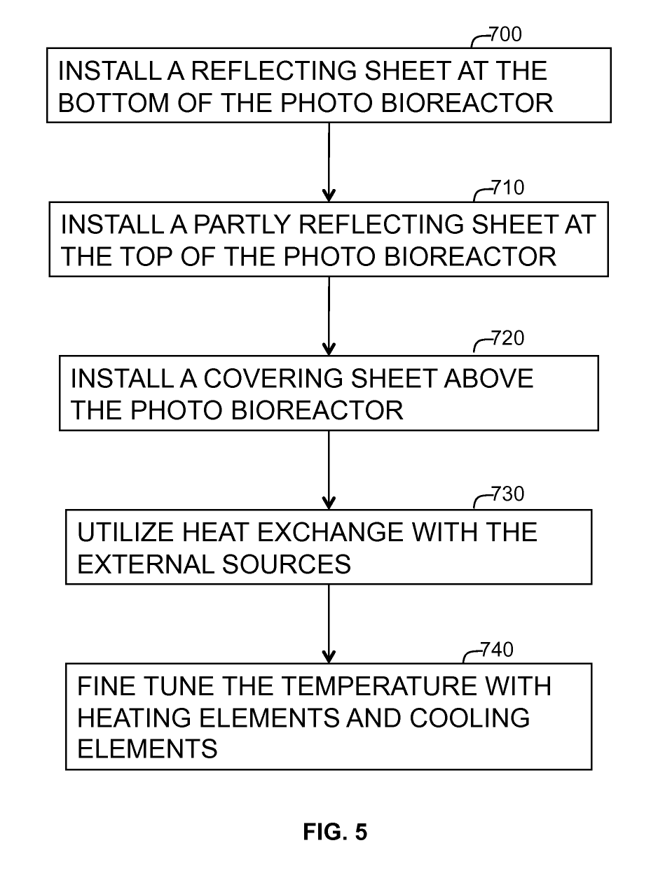

FIG. 5 is a flowchart illustrating optional stages in a stage of sustaining and regulating an algae culture in photo bioreactors (stage 500), according to some embodiments of the invention. The stage of sustaining and regulating an algae culture in photo bioreactors (stage 500) may comprise any of the following stages or a combination of these stages:
Installing a reflecting sheet at the bottom of the photo bioreactor (stage 700);
Installing a partly reflecting sheet at the top of the photo bioreactor (stage 710);
Installing a covering sheet above the photo bioreactor (stage 720);
Utilizing heat exchange with the external sources (stage 730); and
Fine tuning of the temperature with heating elements and cooling elements connected to the photo bioreactor (stage 740).

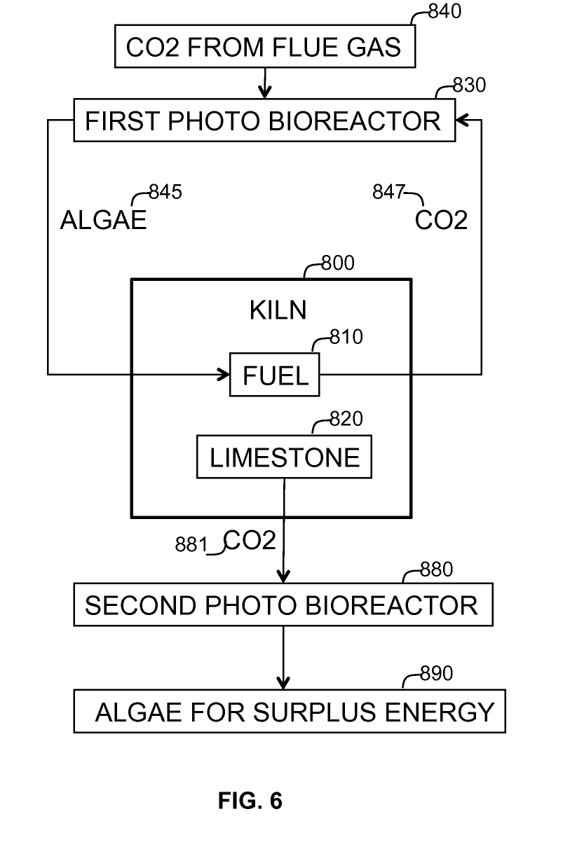

FIG. 6 is a block diagram illustrating a system for producing energy by growing algae in a large excess utilizing CO₂ emitted during the production of clinker and quicklime, according to some embodiments of the invention. The system comprises a kiln 800 comprising a fuel 810 chamber for heating kiln 800 and limestone 820 that is burnt into clinker and quicklime. Fuel 810 comprises algae 845 for a first photo bioreactor 830, which receives CO₂ released by burning algae 845, as well as from other sources such as CO₂ from flue gas 840 e.g. from desalination factories, electricity factories etc. CO₂ 881 released from limestone 820 is fed into a second photo bioreactor 880 which produces a surplus of algae 890 utilized to produce surplus energy.

The rise in the concentration of CO₂ in the atmosphere indicates that the rate with which the biosphere produces carbon compounds is slower than the rate with which carbon compounds are consumed. This low productivity of the biosphere is the result of the low CO₂ concentration in the atmosphere and an adaptation of the existing ecological system to this low concentration.

One way to meet the requirements for carbon compounds is to establish a new artificial ecological system with higher productivity. This ecological system may be based on high growing photosynthetic organisms and high concentration of CO₂ that can support higher growth rate. There is only one source of enough CO₂ for this purpose and that is calcite (CaCO₃) and dolomite (MgCO₃).

Almost all of the CO₂ on the surface of world had been deposited as limestone and dolomite. The carbon in the limestone is not utilizable by the cells due to the low solubility of limestone and the negative free energy of the reaction between calcium and bicarbonate ions. Limestone is an endless resource of carbon for useful energy production. Fortunately the energy required to release the CO₂ from limestone is not too high, and the direction of the process can be reversed by the sunlight energy captured by the photosynthetic cells. The by product and by benefit of this process is quicklime and cement, which are produced by sunlight energy (mediated by cells without any energy cost).

According to some embodiments of the invention, the process is cyclic, continuous, close to the carbon source of the system (CO₂) with high product yield and a very high yield of light energy converted to chemical energy. Some of the advantages of the system are:
1. Cement and quicklime production has been one of the most energy intensive industries in the world (about 40% of the production cost). In this system the energy is supplied by the sun.
2. Cement and quicklime production are very heavy air polluting industries by CO₂—About 9% of the total CO₂ emitted yearly to the atmosphere. In this system all or almost all of this CO₂ is sequestrated and used to grow continuously additional cycles of microalgae cells. Advantages of the proposed system are:
   i. The materials are recycled (CO₂, sea water);
   ii. Cheap equipment and materials;
   iii. Cheap operation cost;
   iv. High concentration of algae and therefore higher yield than any available system;
   v. Self Supplying Energy; and
   vi. Mass Production.

According to some embodiments of the invention, most of the heat energy released during cell burning is produced by 2 reactions: Oxidation of hydrogen atoms to water, in which ΔH°=121 KJ/mol Hydrogen atoms. 100 g of cells contain by mass about 8 g of hydrogen (8%). By burning 100 g of cells the heat energy released by hydrogen oxidation is about 121 KJ/g hydrogen·8 g=968 KJ, or about 9.68 KJ/g hydrogen. The energy (ΔH°f (released during carbon oxidation to CO₂ is about 394 KJ/mol. The atomic mass of carbon is 12 g/mol. By dividing the molar energy released by burning of carbon by it's atomic mass, the value of the heat energy released by burning 1 g of carbon is 32.8 KJ/g. About 50% of the cell mass is carbon. When burning 100 g of cells, the heat energy released from carbon oxidation is 50 g Carbon 32.8 KJ/g=1642 KJ, or about 16.42 KJ/g cells. Hydrogen and carbon contributes about 55-60% of the dried cell mass, and by burning them the heat energy produced is about (16.42+9.68) KJ/g cells=26.1 KJ/g cells. Taking into account oxidation of other elements contained in the cell—the heat energy produced is about 30-35% KJ/g cells. Algae cells are thus comparable to anthracite coal with 23-33 KJ/g.

According to some embodiments of the invention, the system and method utilize cheap and non-polluting quicklime and cement (CaO). The proposed invention includes energy production by sunlight energy with micro-algae cells. Calcium oxide is usually made by the thermal decomposition of materials such as limestone, that contain calcium bicarbonate (CaCO₃); mineral, name calcite, according to the equation CaCO₃+Heat→CO₂+CaO. When the temperature is between 800-1200 Degrees Celsius, the product is quicklime known also as burnt lime. At 1400 Celsius Degrees the product is clinker. The main operational cost of quicklime and cement production is the energy (about 50%). The raw material of cement production (Lime Stone) is very cheap, only a few percents of the overall operational costs. The kiln is fueled with dry micro algae cells.

According to some embodiments of the invention, the proposed system is cyclic and closed for the carbon source. All CO₂ released by burning the micro algae cells and during calcium oxide production is collected and flowed into the photo bioreactor to enable additional cycles of cell growth. Limestone (calcite and dolomite) is an excellent source of CO₂ for photosynthesis. During CaO production a huge amount of CO₂ is released from the limestone. 1 mol of CaCO₃ decompose to 1 mol of CaO (M=46 g/mol) and 1 mol of CO₂ (M=44 g/mol). Each ton of limestone decompose to about half ton of quicklime and about half tone of CO₂.

According to some embodiments of the invention, according to various references, the energy required to produce 1 ton CaO or 1 ton cement is about 4,000-4,600 MJ, or the energy required to produce 1 gram of CaO is about: 4.6 KJ/g. The molecular mass of CaO is (20+16) g/mol=36 g/mol. The energy required to produce 1 mol of CaO is: CaO KJ/g 4.6×36 g/mol=165.6 KJ/mol CaO. If by burning 1 gram of cells, the amount of heat energy produced is 26.1 KJ/g cell. In order to produce 1 mol of CaO the amount of cells that have to be burned is 165.6 KJ/26.1 KJ/g cells=6.34 g cells. Assuming that the carbon content of the cells is 50%, by burning 6.34 gram cells, about 3.17 gram carbon is oxidized to CO₂, or 0.26 moll CO₂, as can be seen in: 3.17 g carbon/12 g carbon/mol=0.26 mol CO₂. The amount of CO₂ emitted from the limestone (calcite and dolomite) is much higher than the amount of CO₂ that was produced by burning cells to release this CO₂ from the limestone. Approximately 3.85 folds: 1 mol CO₂ from limestone/0.26 mol CO₂ from cells=3.85. By supply of this amount of CO₂ (released from limestone) to additional photo bioreactors combined with the system, an additional amount of new cells. This amount of new cells is greater then the amount of the cells burned to release the CO₂ from she limestone by a factor of 3.85 fold. Taking into account that the CO₂ in the flue gas completely (or almost completely) recycled, the amount of the new cells is higher by a factor of about 4.85 fold compared to the original amount of burnt cells.

According to some embodiments of the invention, the rate of energy conversion (Productivity-P) from electromagnetic energy of sunlight to a chemical energy accumulated as organic compounds of micro algae cells is given by the equation: P=YμX, wherein Y is ratio (coefficient) between the heat energy produced by burning a certain amount and the mess of these cells (ΔEH/ΔX). Although cell densities of 2-5 g cell/liter have been reported, let's assume a cell density of a modest value of 1 g cells/liter, doubling time (g) of 6 hours, and Y of 26 KJ/g cell. μ=Ln2/g=Ln2/6 h=0.11/h. With these values the rate of energy accumulation per 1 liter of cell suspension is equal to:

P=26 KJ/g cells 0.11/h·1 g cell/liter=2.6 KJ/liter. Let's take 1 square meter of photo bioreactor as described above with a depth of 30 cm growth medium. The volume of cells suspension under this square meter is 300 liters and the productivity for 1 m² is 2.6 KJ per hour/h 300 L=780 kJ/m² h. Assuming that the heat of combustion of gasoline is 42 KJ/g, this productivity is equal to: 780 kJ/m² h/42 KJ/g gasoline=18.6 g gasoline/m² h. Taking into account that this energy is accumulated only at day light, and taking an average value of 10 hours of light per 24 h—the daily energy productivity of 1 m² is about 7800 KJ/m² per day. If the energy required for production of 1 Kg of cement is about 5000 KJ, the daily yield of 1 Kg of cement can produce 1.6 Kg of cement. The area required to produce 1 ton of cement per day is about 640 m².

According to some embodiments of the invention, the method comprises recycling of the CO₂ in the flue gas according to the following material balance calculation. The burnt carbons supplied by the fuels (micro algae or fossil) are released as CO₂ in the flue gases. The concentration of CO₂ in the exhaust gas of engines or power plant varies between 3% when the fuel contains high hydrogen content (hydrocarbon) and 15% when the fuel is coal (close to 100% carbon). Since cells contain about 50% carbon, it is assumed that the exhaust gas of burnt cell contains an average value of 8-9% of CO₂. Hence, most of the volume of the exhaust gases is not CO₂ but other gasses. Since most of the volume of the flue gas is not CO₂, and the space in the photo bioreactor is limited, most of the exhaust gas must be released from the photo bioreactor with part of the CO₂ in the exhaust gas, and cannot be recycled by the micro algae in the growth medium. The concentration of the CO₂ is kept by controlled supply rate of flue gas, with 8-9% CO₂ concentration flow into the photo bioreactor. At the same time equal flow (same volume) from the bio reactor, with CO₂ concentration of 1%. This implies that most of the CO₂ in the flue gas (about 80-90%) is sequestrated directly by the growing cells. The flow with the remained 1% CO₂ that leaves the Photo bioreactor is repeatedly bubbled through concentrated aqueous solution of ammonia (NH₃) to produce ammonium bicarbonate (NH₄CO₃). Experiments show that more than 90% of the CO₂ react with the ammonia. Thus, the flue gas that is released to the atmosphere from the ammonia tank contains only about 0.1-0.2% CO₂. The mixture of ammonia and ammonium bicarbonate is than supplied to the cells in the photo bioreactor (and to other photo bioreactors combined with the system (see below) as a nitrogen source. As can be seen by the calculation above, most of the CO₂ (more than 98%) is recycled directly or as ammonium bicarbonate. If the CO₂ concentration in the gaseous phase is reduced from 1% to 0.5%, 0.25% or even 0.1%, the growth rate is still equal or close to maximum, but the sequestration of CO₂ from the flue gas is equal or very close to 100%. The rest amount of CO₂ (completion of 100%), if necessary, will be supplied by a small stream of pure CO₂ released from calcite during calcium oxide production.

According to some embodiments of the invention, the following relates to the calculation of energy recovery in the system. 100 g cell contain about 4 mol of carbon (45-50% carbon by mass, M=12 g/mol carbon), and about 1 mol of Nitrogen (14-15% by mass, M=14 g/mol nitrogen). The carbon is up taken by the cell as CO₂ (bicarbonate ions). The production of 1 mol of CO₂ from limestone requires about 150-180 KJ/mol CO₂ of heat energy (the heat energy consumption of the process). Production of 100 g cell requires about 170 KJ/mol 4 mol carbon/100 g cell=680 KJ/100 g cell. Or, for production of 1 gram of cells: 6.8 KJ/g cell. The nitrogen is up taken by the cells as ammonia (NH₄ ions). Ammonia is produced by Haber-Bosch process at high temperature (400-500° c.) and high pressure (300-1000 at). Although the reaction between nitrogen and hydrogen is very exothermic (ΔHf°=−94 kj/mol Ammonia) the activation energy is very high, and this is one of the most intensive energy consuming industrial process. According to various references, the specific energy consumption of ammonia production varies between 40 kj/g ammonia to 28 kj/g ammonia. Taking an average value of 35 kj/g ammonia, the energy consumed for production of 1 mol ammonia is about: 35 kj/g ammonia 17 g/mol ammonia=595 kj/mol ammonia. This is the energy required to produce the ammonia for growth of 100 g cell, and for 1 g cell the amount is: 5.95 kj/g cell. The nitrogen of the combusted cells also flow into the bioreactor as NOx compounds. These compounds are easily up taken by the cells. The values used in the above calculations and the energy input for this ammonia production are adequate to photo bioreactors which are supplied with CO₂ emitted from limestone. In photo bioreactors supplied with flue gas, the required amount of ammonia is supposed to be much smaller. Cell contains about 20% oxygen supplied by CO₂ or as molecular oxygen. The 8% hydrogen is supplied from water by sunlight energy. All other elements are up taken as salt ions and the energy input for their supply is negligible. The energy input for the cell production (growth) is the sum of energy consumed for the production of the material they need for growth and the operational energy of the photo bioreactor. But, for a 1 g of cell the operation energy can also be considered as very small. If the carbon source is decomposition of limestone and the nitrogen is supplied as ammonia produced by Haber-Bosch process, the specific energy input for production of 1 g cell can be estimated as: 12-13 KJ/g cell. This specific energy input is approximately half of the specific combustion energy content of the cells which is estimated as: 25-30 KJ/g cell. This difference between the input energy and energy content of the cells is a gift of the sun. The above calculations indicate that the energy content of the cells is large enough to release from limestone the amount of CO₂ needed for their growth, to produce the amount of ammonia needed for their growth, and a substantial amount of energy is still left to fuel other energy consuming processes. These calculations also indicate that energy problems of the globe are not quantitative as long as the sun arises and enough area is available to absorb sunlight by the photosynthetic cells. The problems are appropriate technology and cost. I suggest here some solutions for both aspects of these energy problems. When oil is extracted from cells (about 50% of the cell dry weight) and is trans-esterified, too much energy is consumed for the production of the fuel (biodiesel), and too large part of the energy content of the cell is not extracted and lost. Consequently, no net energy (or only small amount of energy) is left. Therefore it is preferred to fuel kilns (for CO₂ production and other processes) with cells instead of fossil fuel. Most of the air is nitrogen, more than 79%, so there is no lack of this resource. On the other hand, the concentration of CO₂ in the air is only 0.03%, too low to enable “bioenergy” production in appropriate rate. If it is wished to reduce consumption of fossil fuels, or to save them for other utilities such as row materials for petro chemical industries, another source of CO₂, except fossil fuel, must be found. Almost all of the CO₂ on the surface of world had been deposited as limestone. The carbon in the limestone is not utilizable by the cells due to the low solubility of limestone and the negative free energy of the reaction between calcium and bicarbonate ions. Limestone is an endless resource of carbon for useful energy production. Fortunately the energy required to release the CO₂ from limestone is not too high, and the direction of the process can be reversed by the sunlight energy captured by the photosynthetic cells. The by product and by benefit of this process is quicklime and cement, which are produced by sunlight energy (mediated by cells) without any energy cost.

According to some embodiments of the invention, the quantity of CO₂ released from burning algae and limestone may allow growing 4-5 times the algae burnt. The system actually produces clinker from limestone utilizing solar energy in the mediation of algae. The system accumulated biofuel in the form of algae to an amount exceeding manifolds the consumed amount of fuel. The source of carbon is CO₂ from limestone and the source of energy is the sun. The energy surplus may be consumed to produce electricity, water desalination, etc. The system does not emit CO₂ but recycles carbon and nitrogen sources. The system may increase its energy production geometrically, as long as area and limestone are available. The system is simple and requires cheap materials, yet produces unlimited amount of energy, thus reducing fuel prices.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention can be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Those skilled in the art will envision other possible variations, modifications, and applications that are also within the scope of the invention. Accordingly, the scope of the invention should not be limited by what has thus far been described, but by the appended claims and their legal equivalents.

What is claimed is:

1. A system for energy production comprising:
at least one kiln for burning limestone and dolomite into clinker;
at least two photo bioreactors for growing algae;
a first pipework for feeding CO₂ from the at least one kiln to a first the photo bioreactor;
a second pipework for feeding CO₂ released from the burning limestone and dolomite to a second the photo bioreactor;
a harvester for taking algae out of the photo bioreactors; and
an energy producing module for producing energy from the biomass of the algae taken out of the photo bioreactors,
wherein CO₂ from the at least one kiln is used to intensify the growth of the algae in the photo bioreactors.

2. The system of claim 1, wherein at least one of the photo bioreactors comprises at least one temperature regulating element.

3. The system of claim 2, wherein the at least one temperature regulating element comprises at least one of: heating coils, cooling flanges, reflective material on the bottom of the photo bioreactor, reflective material on top of the photo bioreactor, covering material, ground contact.

4. The system of claim 1, wherein substantially all CO₂ released by burning limestone and dolomite into clinker in the at least one kiln is introduced into the photo bioreactor.

5. The system of claim 1, further comprising at least one analyzer connected to a control system, the at least one analyzer arranged to keep the CO₂ concentration in the at least one photo bioreactor at an optimal level.

6. The system of claim 1, further comprising a bubbler for introducing CO₂ into at least one of the photo bioreactors.

7. The system of claim 1, wherein the harvester comprise:
at least one pump for pumping water out of at least one of the photo bioreactor;
at least one filter for extracting algae out of the pumped water; and
at least one drier for drying the extracted algae.

8. The system of claim 1, wherein the energy producing module comprises the at least one kiln.

9. A method of producing energy comprising:
sustaining and regulating an algae culture in at least two photo bioreactors;
introducing CO₂ from at least one kiln arranged for burning limestone and dolomite into clinker;
harvesting algae from the bioreactors; and
producing energy from the biomass of harvested algae.

10. The method of claim 9, further comprising keeping a CO₂ concentration at an optimal level utilizing a analyzer connected to the control system.

11. The method of claim 9, further comprising utilizing a CO₂ concentration of around 3% in the air supplied to the photo bioreactors.

12. The method of claim 9, wherein the introducing CO₂ from at least one kiln comprises bubbling CO₂ rich air into at least one of the photo bioreactor.

13. The method of claim 9, wherein the sustaining and regulating an algae culture in photo bioreactors comprises at least one of: installing a reflecting sheet at the bottom of the photo bioreactor, installing a partly reflecting sheet at the top of the photo bioreactor, installing a covering sheet above the photo bioreactor, utilizing heat exchange with the external sources, and fine tuning of the temperature with heating elements and cooling elements connected to the container.

14. The method of claim 9, wherein the harvesting algae from the bioreactors comprises:
pumping water out of photo bioreactor;
filtering the water to extract algae from it;
drying extracted algae; and
burning the dried extracted algae, and
wherein harvesting rate is related to algae growth rate and the flow rates of water, nutrients and algae to and from at least one the photo bioreactor.

15. The method of claim 9, wherein the drying extracted algae comprises utilizing hot air from an external process, and wherein the air may be recycled.

16. The method of claim 9, wherein the burning algae is carried out in the at least one kiln.

17. The method of claim 9, further comprising utilizing heat from the burning the dried extracted algae to at least one of: producing electricity via steam, desalinating water, satisfying peaks in electricity consumption, and performing an industrial process.

18. The method of claim 9, further comprising feeding at least part of CO₂ and heat resulting from the burning the dried extracted algae back into at least one of the photo bioreactors.

19. The method of claim 9, further comprising feeding at least part of the water extracted during the drying the extracted algae back into at least one of the photo bioreactors.

20. The method of claim 9, wherein the introducing CO₂ from at least one kiln comprises introducing substantially all CO₂ released by burning limestone and dolomite into clinker in the at least one kiln.
